(19) United States
(12) Patent Application Publication
(10) Pub. No.: US 2007/0014790 A1
(43) Pub. Date: Jan. 18, 2007

(54) Transcription control factor zhx3

(75) Inventors: Kaoru Miyamoto, Sakai-gun, Fukui (JP); Kazuya Yamada, Fukui (JP)

Correspondence Address: BUCHANAN, INGERSOLL & ROONEY PC, POST OFFICE BOX 1404, ALEXANDRIA, VA, 22313-1404, US

(73) Assignee: Japan Science And Techology Agency, Saitama (JP)

(21) Appl. No.: 10/534,424

(22) PCT Filed: Jul. 18, 2003
(86) PCT No.: PCT/JP03/09164

§ 371 Date: May 10, 2005

(30) Foreign Application Priority Data
Dec. 18, 2002 (JP) 2002-366512

(51) Int. Cl. A61K 39/395; C12N 9/12; C07K 14/705

(52) U.S. Cl. 424/143.1; 530/350; 530/388.22; 435/194

(57) ABSTRACT

To determine the biological role of ZHX1, found previously, which acts as a transcriptional repressor, the inventors conducted a search of ZHX1—interacting proteins using a yeast two-hybrid system. Molecular cloning and determination of the nucleotide sequence of the full-length cDNA encoding a novel protein revealed a novel protein with SEQ ID NO: 1. The protein (ZHX3), like ZHX1, contains two zinc-finger (Znf) motifs and five homeodomains (HDs) and has a transcriptional repressor activity.

### FIELD OF THE INVENTION

This invention relates to protein acting as a transcriptional suppressor, more specifically to a transcriptional regulator ZHX3 for genes specific to hepatoma.

### PRIOR ART

Transcription of pyruvate kinase (PK) M gene and type II hexokinase (HK II) gene, genes for glycolytic pathway, is induced in hepatoma cells, but silent in normal hepatocytes. The common transcription factor for both genes is nuclear factor-Y (NF-Y), however, there are no difference in the expression of NF-Y between normal hepatocytes and hepatoma cells. Therefore, some interacting partners of NF-Y may be responsible to the gene expression specific to hepatoma cells.

Running a search for proteins interacting with A subunit (NF-YA), an important subunit of NF-Y, the inventors cloned ZHX1, which consists of 873 amino acids and contains two zinc-finger motifs (Znf) and five homeodomains (HD) (Yamada, K., Osawa, H., and Granner, D. K. (1999) FEBS Lett. 460, 41-45). ZHX1 belongs to the Znf class of the homeobox protein superfamily and the amino acid sequence between 272 and 564 that contains the HD1-HD2 region of human ZHX1 is required for an interaction with the N-terminal glutamine-rich AD of NF-YA (Yamada, K., Osawa, H., and Granner, D. K. (1999) FEBS Lett. 460, 41-45). Northern blotting of ZHX1 revealed that the ZHX1 transcripts were expressed ubiquitously (Yamada, K., Printz, R. L., Osawa, H., and Granner, D. K. (1999) Biochem. Biophys. Res. Commun. 261, 614-621; Hirano, S., Yamada, K., Kawata, H., Shou, Z., Mizutani, T., Yazawa, T., Kajitani, T., Sekiguchi, T., Yoshino, M., Shigematsu, Y, Mayumi, M., and Miyamoto, K. (2002) Gene 290, 107-114). The human ZHX1 gene is located on chromosome 8q, between markers CHLC.GATA50B06 and CHLC.GATA7G07 (Yamada, K., Printz, R. L., Osawa, H., and Granner, D. K. (1999) Biochem. Biophys. Res. Commun. 261, 614-621). The inventors reported that ZHX1 functions as a transcriptional repressor and is localized in the nuclei (Yamada, K., Kawata, H., Matsuura, K., Shou, Z., Hirano, S., Mizutani, T., Yazawa, T., Yoshino, M., Sekiguchi, T., Kajitani, T., and Miyamoto. K. (2002) Biochem. Biophys. Res. Comm. 279, 368-374).

By the way, a nucleotide sequence (GenBank/XM 029734) containing the sequence encoding a novel transcriptional regulator (ZHX3) discovered by the inventors and a part of the sequence (DDBJ/AB007855) have been registered to the parenthetic database. However, the former has been registered as a putative protein by linking portions of the sequence scattered on the database and is suggestive of coding a certain protein. Also, the latter is a sequence of cloned partially without functional analysis. Namely, transcriptional repressor activity of the protein encoded by these disclosed nucleotide sequences has neither been known nor been guessed based on the disclosed information.

### PROBLEMS TO BE SOLVED BY THE INVENTION

To determine the biological role of ZHX1, the inventors examined the issues of whether ZHX1 interacts with protein(s) other than NF-YA and regulates gene transcription. Using a yeast two-hybrid system, the inventors conducted a search for ZHX1-interacting proteins in rat liver and ovarian granulosa cell cDNA libraries.

### MEANS TO SOLVE THE PROBLEMS

As the result of the search, nuclear proteins, such as ZHX1, transcription co-factors, DNA-binding proteins, zyxin, and androgen-induced aldolase reductase, 11-19 lysine-rich leukemia gene, as well as other unknown proteins, were cloned and a novel protein was found. Molecular cloning and determination of the nucleotide sequence of the full-length cDNA encoding the novel protein revealed that it consists of the 956 amino acid residues (SEQ ID NO: 1) and contains, like ZHX1, two zinc-finger (Znf) motif and five homeodomains (HDs).

The inventors concluded that the protein forms the ZHX family with ZHX1 and denoted it ZHX-3. It is deemed that the proteins of ZHX family may be involved in gene regulation by forming homodimers or heterodimers by interacting with each other.

ZHX3 not only dimerizes with both ZHX1 and ZHX3, but also interact with the activation domain (AD) of the NF-YA. Further analysis revealed that ZHX3 is a ubiquitous transcriptional repressor that is localized in nuclei and functions as a dimer.

Therefore, the present invention is a protein or a peptide having a transcriptional repressor activity, comprising an amino acid sequence of SEQ ID NO: 1, an amino acid sequence comprising a deletion, substitution or addition of one or several amino acids with respect to the amino acid sequence of SEQ ID NO: 1, the functional domain of an amino acid sequence of SEQ ID NO: 1, or an amino acid sequence comprising a deletion, substitution or addition of one or several amino acids with respect to the functional domain of an amino acid sequence of SEQ ID NO: 1.

The similarities in the amino acid sequences of ZHX3 between human and mouse is 85.3% and that between human and rat is 87.3%. The amino acid sequence of rat ZHX3 is shown in SEQ ID NO: 2. The rat-type amino acid sequence corresponds to amino acids 114-642 of human ZHX3 amino acid sequence. Therefore, the proteins comprising amino acid sequence with more than 85% similarity to ZHX3 amino acid sequence (SEQ ID NO: 1) could be ascribed to the protein with the same transcriptional repressor activity to human ZHX3.

Therefore, the present invention can be interpreted as a protein or a peptide having an amino acid sequence of SEQ ID NO: 1, or a protein or a peptide having an amino acid sequence with at least 85% similarity to SEQ ID NO: 1 or the functional domain of the amino acid sequence of SEQ ID NO: 1 or the amino acid sequence with at least 85% similarity to said functional domain, and having a transcriptional repressor activity.

As shown by the examples to be hereinafter described, the sequence of amino acids 303-502 of the amino acid sequence of SEQ ID NO: 1 is the domain involved in the function of transcriptional repressor. Therefore, the functional domain of the amino acid sequence of SEQ ID NO: 1 preferably consists of amino acids 303-502 of SEQ ID NO: 1.

Since hepatoma cells are conducting energy metabolism depending preferentially on an enhanced glycolysis, ZHX3 may repress, in normal hepatocytes, the expression of isoenzyme gene family specific to hepatoma cells, and the expression of ZHX3 may be lowered during malignant transformation or ZHX3 protein may be inactivated by modification. Therefore, once agents to detect ZHX3 or drugs to regulate the function are developed, these can be applied to diagnosis and treatment of hepatoma.

Therefore, the present invention is a drug agent to repress transcription, comprising said protein or peptide as an effective component. Furthermore, the invention is either of said protein or peptide to repress transcription of genes expressed specifically in hepatoma cells.

The genes could be type II hexokinase or pyruvate kinase M gene. Also, the invention is a therapeutic product for hepatoma comprising the protein or peptide as an effective component. Moreover, the invention is a method to treat decease, especially hepatoma, resulting from defection of the transcriptional repressor, by using the protein or peptide.

Additionally, the invention is an antibody specific to either of said protein or peptide. Also, the invention is a screening agent to screen drug agents with transcriptional repressor activity, comprising the antibody as an effective component.

### BRIEF DESCRIPTION OF THE DRAWINGS

FIG. 1 shows a comparison of deduced amino acid sequences of human ZHX3 with human ZHX1. The amino acid sequences of ZHX3 and ZHX1 are compared. Two Znf and five HD motifs were shown by plus signs and underlines, respectively. Asterisks indicate the similarity of amino acid sequence. Dashed lines show gaps when corresponding amino acids are absent in each protein. The deduced amino acid sequences of clone G58, G23 and KIAA0395 corresponding to amino acid sequence between 114 and 642, between 242 and 615 and between 495 and 956 of human ZHX3, respectively, are included in FIG. 1.

FIG. 2 shows the tissue distribution of human ZHX3 mRNA. Each lane contains 2 μg of poly(A)⁺-RNA isolated from indicated tissues. Size markers are shown on the left in kb. Lane 1, heart; lane 2, brain; lane 3, placenta; lane 4, lung; lane 5, liver; lane 6, skeletal muscle; lane 7, kidney; lane 8, pancreas; lane 9, spleen; lane 10, thymus; lane 11, prostate; lane 12, testis; lane 13, ovary; lane 14, small intestine; lane 15, colon; lane 16, leucocyte.

FIG. 3 shows the identification of the minimal heterodimerization domain between ZHX1 and ZHX3 using the yeast two-hybrid system, GST pull-down assays. A schematic representation of human ZHX1 and the GAL4 AD-ZHX1 fusion constructs is depicted on the left. Znf and HD indicate zinc-finger motif and homeodomein, respectively. The + and − symbols indicate increased and unchanged levels of β-galactosidase activity, respectively, compared with a yeast harbouring a combination of pDBD-G23, expressing amino acids 242-488 of human ZHX3 fused to the GAL4 DBD, and pACT2.

FIG. 4 shows the identification of the minimal heterodimerization domain between ZHX1 and ZHX3 using the yeast two-hybrid system, GST pull-down assays. A schematic representation of human ZHX3 and the GAL4 AD-ZHX3 fusion constructs is depicted on the left. Znf, HD and E indicate zinc-finger motif, homeodomein, a glutamic-acid-rich region, respectively. + and − indicate increased and unchanged levels of β-galactosidase activity, respectively, compared with a yeast harbouring a combination of pDBD-ZHX1 (1-873), expressing the entire coding region of human ZHX1 fused to the GAL4 DBD, and pACT2.

FIG. 5 shows the identification of the minimal heterodimerization domain between ZHX1 and ZHX3 using the yeast two-hybrid system, GST pull-down assays. In vitro-translated, ³⁵S-labelled full-length human ZHX1 or ZHX3 was incubated with Sepharose beads containing bound GST alone (lanes 1, 5 and 8) or amino acids 242-615 of ZHX3 (lane 3), or the entire coding sequences of human ZHX3 (lane 6) or ZHX1 (lane 9) protein fused to GST. The beads were washed thoroughly and the bound protein was eluted and analysed by SDS/PAGE (10% gel). Interaction signals were detected by an autoradiography. Lanes 2, 4 and 7, 10% of the protein added to the reactions shown in the other lanes were loaded.

FIG. 6 shows the identification of the minimal homodimerization domain of ZHX3 using the yeast two-hybrid system, GST pull-down assays. A schematic representation of human ZHX3 and the GAL4 AD-ZHX3 fusion constructs is depicted on the left. Znf, HD and E indicate zinc-finger motif, homeodomein, a glutamic-acid-rich region, respectively. The + and − symbols indicate the same as described for FIG. 3.

FIG. 7 shows the identification of the minimal homodimerization domain of ZHX3 using the yeast two-hybrid system, GST pull-down assays. In vitro-translated, ³⁵S-labelled full-length human ZHX3 was incubated with Sepharose beads containing bound GST alone (lanes 2) or the entire coding sequences of human ZHX3 (lane 3) protein fused to GST. Procedures followed were the same to those described for FIG. 5. Lane 1, 10% of the protein added to the reactions shown in the other lanes were loaded.

FIG. 8 shows the interaction domain mapping between NF-YA and ZHX3 using the yeast two-hybrid system. A schematic representation of human ZHX3 and the GAL4 AD-ZHX3 fusion constructs are depicted on the left. Znf, HD and E indicate zinc-finger motif, homeodomein, a glutamic-acid-rich region, respectively. + and − indicate increased and unchanged levels of β-galactosidase activity, respectively, compared with a yeast harbouring a combination of pYA1-269, expressing the NF-YA AD fused to the GAL4 DBD, and pACT2.

FIG. 9 shows the interaction domain mapping between NF-YA and ZHX3 using the yeast two-hybrid system. Schematic diagram of NF-YA and its deletion mutants fused to the GAL4 DBD are depicted on the left. Q and S/T indicate glutamine-rich and serine/threonine-rich regions, respectively. SID and DBD indicate the subunit-interaction domain and DNA binding domain, respectively. The + and − symbols indicate increased and unchanged levels of β-galactosidase activity, respectively, compared with a yeast harbouring a combination of pDBD and pAD-ZHX3 (242-488), expressing amino acids 242-488 of human ZHX3 fused to the GAL4 AD.

FIG. 10 shows that ZHX3 is a transcriptional repressor. HEK293 cells were co-transfected with 2 ng of the pRL-CMV, 50 ng of the simian virus 40 (SV40) promoter-directed expression vector and 100 ng of the 5×GAL4-pGL3 Control or pGL3-Control reporter plasmid. pSG424 and pGAL4-ZHX3 (1-956) express GAL4 DBD alone and the entire coding sequence of human ZHX3 fused to the GAL4 DBD, respectively. A value of 100% was assigned to the promoter activity for the reporter plasmid in the presence of 50 ng of pSG424.

FIG. 11 shows that ZHX3 is a transcriptional repressor. HEK293 cells were co-transfected with 2 ng of the pRL-CMV, 50 ng of the SV40 promoter-directed expression vector, 100 ng of the 5×GAL4-pGL3 Control reporter plasmid and the indicated amount of pCMV-ZHX1 (242-432) expression plasmid. The total amount of plasmid (202 ng) was adjusted by the addition of the pcDNA3.1His-C2, if necessary. A value of 100% was assigned to the promoter activity of the reporter plasmid in the presence of 50 ng of pSG424 and 50 ng of pcDNA3.1His-C2. 48 h after transfection, cells were harvested and both firefly and sea pansy luciferase activities were determined. Firefly luciferase activities were normalized by the sea pansy luciferase activities in all experiments. Each column and bar represents the mean±S.D. from at least five transfection experiments.

FIG. 12 shows the minimal repressor domain of ZHX3. The pSG424 and various pGAL4-ZHX3 constructs express GAL4 DBD alone and various deletion mutants of human ZHX3 fused to the GAL4 DBD. Conditions were the same as described for FIG. 10. Each column and bar represents the mean±S.D. from at least five transfection experiments.

FIG. 13 shows subcellular localization of ZHX3 in HEK293 cells and determination of NLSs. Expression plasmids (300 ng) encoding GFP alone or various truncated ZHX3 proteins fused to the C-terminus of GFP were transfected into HEK293 cells. At 48 h after transfection, the subcellular localization of GFP fusion proteins was observed. Constructs are given at the top of each panel.

FIG. 14 shows schematic diagram of the functional domains of human ZHX3. Znf, zinc-finger motif; HD, homeo domai; E, glutamic acid-rich region; DD, dimerization domain; ID, interaction domain with NF-YA; RD, repressor domain; NLS, nuclear-localization signal.

The following examples illustrate this invention, however, these are not constructed to limit the scope of this invention.

### EXAMPLES

In the following examples, the yeast two-hybrid system, pDsRed1-C1, X-α-gal, human testis marathon-ready cDNA, Advantage 2 PCR kit, human Multiple Tissue Northern Blot and Blot II, ExpressHyb hybridization solution and pEGFP-C1 were purchased from Clontech (Palo Alto, Calif., U.S.A.). The pGEX-4T-2, pGEX-5X-1, α-³²PdCTP (111 TBq/mmol), glutathione-Sepharose 4B and [³⁵]methionine (37 TBq/mmol) were purchased from Amersham Pharmacia Biotech (Cleveland, Ohio, U.S.A.). HEK293 cells, a human embryonic kidney cell line, were purchased from the American Type Culture Collection (Manassas, Va., U.S.A.). Trizol reagent, Superscript II, pcDNA3.1His-C plasmid and LIPOFECTAMINE PLUS were purchased from Invitrogen (Groningen, Netherlands). ExTaq DNA polymerase, pT7Blue-T 2 vector and BcaBest DNA-labelling kit were obtained from TaKaRa Biomedicals (Kyoto, Japan). pGEM-T Easy vector, T7 TNT Quick-coupled transcription/translation system, pGL3-Control, pRL-CMV and dual-luciferase assay system were purchased from Promega (Madison, Wis., U.S.A.). The Big Dye terminator FS cycle sequencing kit was purchased from Applied Biosystems Japan (Tokyo, Japan). TOPP3 cells were obtained from Stratagene (La Jolla, Calif., U.S.A.). Qiagen plasmid kit were purchased from Qiagen (Hilden, Germany).

The followings are the experimental procedures used for the present examples.

### Reference Example 1

### Plasmid Construction

The pACT2B1 plasmid has been prepared as described previously (Yamada, K., Osawa, H., and Granner, D. K. (1999) FEBS Lett. 460, 41-45). pAD-G23, a cloned G23 plasmid, was digested with EcoRI/XhoI or SfiI/BglII and each fragment was subcloned into the EcoRI/XhoI sites of the pGEX-4T-2 vector or SfiI/BamHI sites of the pGBKT7 vector to obtain pGST-G23 and pDBD-G23, respectively.

A 250-bp EcoRI/HindIII fragment of pAD-G23 was subcloned into the EcoRI/HindIII sites of the pDsRed1-C1 to produce pDsRed-revereseG23 (HD1). The S-DsRed1C1-HindIII (SEQ ID NO: 3) and the As-DsRed1C1-SalI oligonucleotides (SEQ ID NO: 4) were then annealed, phosphorylated and inserted into the HindIII/SalI sites of the pDsRed1-C1 to obtain pDsRed1-C1E1. An EcoRI/BglII fragment of the pDsRed-revereseG23 (HD1) was subcloned into the EcoRI/BamHI sites of the pDsRed1-C1E1 to produce pDsRed-rZHX3 (HD1). The EcoRI/XhoI fragment of the resultant plasmid was subcloned into the EcoRI/XhoI sites of the pACT2 to produce pAD-ZHX3 (242-488).

pBSII-KIAA0395 was a gift from Dr Takahiro Nagase (Kazusa DNA Research Institute, Chiba, Japan). A 1.2-kb SalI/ApaI fragment of the plasmid was subcloned into the SalI/ApaI sites of the pDsRed1-C1E1 to produce pDsRed-ZHX3 (HD2-5). The BglII/BamHI fragment of the resultant was subcloned into the BamHI site of pACT2B1 to obtain pAD-ZHX3 (498-903).

The pAD-ZHX1 (142-873), pAD-ZHX1 (272-873), pAD-ZHX1 (565-873), pAD-ZHX1 (272-564), pAD-ZHX1 (272-432), pAD-ZHX1 (430-564), pAD-ZHX1 (345-463), pDBD, pYA1-269, pYA1-140, pYA1-112, pYA141-269, pYA172-269 and pYA205-269 were constructed as described previously (Yamada, K., Osawa, H., and Granner, D. K. (1999) FEBS Lett. 460, 41-45; Yamada, K., Printz, R. L., Osawa, H., and Granner, D. K. (1999) Biochem. Biophys. Res. Commun. 261, 614-621).

Total RNA from HEK293 cells was prepared using the TRIZOL reagent according to the manufacturer's protocol. Reverse transcriptase PCRs (RT-PCRs) were performed as described previously with minor modification (Yamada, K., Printz, R. L., Osawa, H., and Granner, D. K. (1999) Biochem. Biophys. Res. Commun. 261, 614-621). PCR conditions were described previously except for the use of the ExTaq DNA polymerase (Yamada, K., Printz, R. L., Osawa, H., and Granner, D. K. (1999) Biochem. Biophys. Res. Commun. 261, 614-621). Combinations of S-hZHX3-ApaI2 (SEQ ID NO: 5) and As-hZHX3-STOP4 (SEQ ID NO: 6), S-hZHX3-NcoI (SEQ ID NO: 7) and As-hZHX3-BsmBI2 (SEQ ID NO: 8), S-hZHX3-Met (SEQ ID NO: 9) and As-hZHX3-NcoI (SEQ ID NO: 10), S-hZHX3-HD1 (SEQ ID NO: 11) and As-hZHX3-HD2 (SEQ ID NO: 12), S-hZHX3-HD1 (SEQ ID NO: 11) and As-hZHX3-1506 (SEQ ID NO: 13), S-hZHX3-1090 (SEQ ID NO: 14) and As-hZHX3-HD2 (SEQ ID NO: 12), S-hZHX3-HD2 (SEQ ID NO: 15) and As-hZHX3-HD2 (SEQ ID NO: 12), S-hZHX3-HD1 (SEQ ID NO: 1) and As-hZHX3-HD1 (SEQ ID NO: 16), and S-hZHX3N (SEQ ID NO: 17) and As-hZHX3N (SEQ ID NO: 18), were used as primers.

These products were subcloned into the pGEM-T Easy vector to give pGEM-T Easy ZHX3 (ApaI/STOP), pGEM-T Easy ZHX3 (NcoI/BsmBI), pGEM-T Easy ZHX3 (Met/NcoI), pGEM-T Easy ZHX3 (HD1-2), pGEM-T Easy ZHX3 (HD1-1506), pGEM-T Easy ZHX3 (1090-HD2), pGEM-T Easy ZHX3 (HD2), pGEM-T Easy ZHX3 (HD1) and pGEM-T Easy ZHX3N, respectively.

GBKT7MCS1 (SEQ ID NO: 19) and GBKT7MCS2 oligonucleotides (SEQ ID NO: 20) were annealed, phosphorylated and subcloned into the EcoRI/BamHI sites of pGBKT7 to give pGBKT7B1. The ApaI/BamHI fragment of the pGEM-T Easy ZHX3 (ApaI/STOP) was subcloned into the ApaI/BamHI sites of the pDsRed-ZHX3 (HD2-5) to give pDsRed-ZHX3 (HD2-STOP). The EcoRI/BsmBI fragment of pGEM-T Easy ZHX3 (NcoI/BsmBI) was subcloned into the EcoRI/BsmBI sites of pDsRed-ZHX3 (HD2-STOP) to give pDsRed-ZHX3 (NcoI-STOP).

S-GBKT7-NdeI (SEQ ID NO: 21) and As-GBKT7-NcoI oligonucleotides (SEQ ID NO: 22) were annealed, phosphorylated and inserted into the NdeI/NcoI sites of pGBKT7B1 to give pGBKT7NEN. A 2-kb NcoI/BamHI fragment of the pDsRed-ZHX3 (NcoI-STOP) was subcloned into the NcoI/BamHI sites of the pGBKT7NEN to produce pGBKT7-ZHX3 (NcoI-STOP). pGEM-T Easy ZHX3 (Met/NcoI) was digested with NcoI and the 960-bp fragment was subcloned into the NcoI site of pGBKT7-ZHX3 (NcoI-STOP) to give pDBD-ZHX3 (1-956). The resultant plasmid was digested with BamHI, blunt-end ligated by the Klenow reaction, and then digested with EcoRI. The 2.9-kb fragment was subcloned into the EcoRI/SmaI sites of the pGEX-5X-1 vector to obtain pGST-ZHX3 (1-956). The EcoRI/XhoI fragment of the pGST-ZHX3 (1-956) was subcloned into the EcoRI/XhoI sites of the pACT2B1 to produce pAD-ZHX3 (1-956). PCRs were carried out using the pDBD-ZHX3 (1-956) as a template with the combination of S-hZHX3HD1 (SEQ ID NO: 11) and As-hZHX3-HD1-Eco (SEQ ID NO: 23) as primers. After digestion with EcoRI, the fragment was subcloned into the EcoRI site of pACT2B1 to give pAD-ZHX3 (303-364).

The pSG424, pSG424B1, 5×GAL4-pGL3 Control and pEGFP-C1E1 plasmids have been described previously. The 2.9-kb EcoRI/BamHI fragment of the pDBD-ZHX3 (1-956) was subcloned into the EcoRI/BamHI sites of the pSG424B1 or pEGFP-C1E1 vector to give pGAL4-ZHX3 (1-956) and pGFP-ZHX3 (1-956), respectively. The BglII/BamHI fragment of the pDsRed-ZHX3 (HD2-5) was subcloned into the BamHI site of pSG424B 1 to give pGAL4-ZHX3 (498-903). The EcoRI/BamHI fragments of the pGEM-T Easy ZHX3 (HD1-2), pGEM-T Easy ZHX3 (HD1-1506), pGEM-T Easy ZHX3 (1090-HD2) and pGEM-T Easy ZHX3 (HD2) were subcloned into the EcoRI/BamHI sites of the pSG424B1 or pEGFP-C1E1 vector to produce pGAL4-ZHX3 (303-555), pGAL4-ZHX3 (303-502), pGFP-ZHX3 (303-555), pGFP-ZHX3 (303-502), pGFP-ZHX3 (364-555) and pGFP-ZHX3 (497-555), respectively. The EcoRI/BamHI fragment of the pGEM-T Easy ZHX3 (HD1) was subcloned into the EcoRI/BamHI sites of the pSG424B1 vector to obtain pGAL4-ZHX3 (303-364).

PCRs were also carried out using pDBD-ZHX3 (1-956) as a template with the combination of S-hZHX3-Met3 (SEQ ID NO: 24) and As-hZHX3-909 (SEQ ID NO: 25), S-hZHX3-Met3 (SEQ ID NO: 24) and As-hZHX3-435 (SEQ ID NO: 26), S-hZHX3-436 (SEQ ID NO: 27) and As-hZHX3-909 (SEQ ID NO: 25), S-hZHX3-Met3 (SEQ ID NO: 24) and As-hZHX3-321 (SEQ ID NO: 28), S-hZHX3-322 (SEQ ID NO: 29) and As-hZHX3-435 (SEQ ID NO: 26), and S-hZHX3-1663 (SEQ ID NO: 30) and As-hZHX3-BsmBI-2 (SEQ ID NO: 31), as primers, and using the pGFP-ZHX3 (303-555) as a template with the combination of S-hZHX3-1090 (SEQ ID NO: 14) and As-hZHX3-1506 (SEQ ID NO: 13) as primers. Amplified DNAs were also subcloned into the pGEM-T Easy or pT7Blue-2 T vector to produce pGEM-T Easy ZHX3 (Met/909), pGEM-T Easy ZHX3 (Met/435), pGEM-T Easy ZHX3 (436/909), pT7Blue-2 T ZHX3 (Met/321), pT7Blue-2 T ZHX3 (322/435), pGEM-T Easy ZHX3 (1663/2022) and pGEM-T Easy ZHX3 (1090/1506), respectively. The EcoRI/BamHI fragments of these plasmids were subcloned into the EcoRI/BamHI sites of the pSG424B1 or pEGFP-C1E1 vector to produce pGAL4-ZHX3 (1-145), pGAL4-ZHX3 (146-303), pGFP-ZHX3 (1-303), pGFP-ZHX3 (1-107), pGFP-ZHX3 (108-145) and pGFP-ZHX3 (146-303), respectively. The EcoRI/BamHI fragment of the pGEM-T Easy ZHX3 (1090/1506) was subcloned into the EcoRI/BamHI sites of the pSG424B1 to produce pGAL4-ZHX3 (364-502). The EcoRI/BsmBI fragment of the pGEM-T Easy ZHX3 (1663/2022) was subcloned into the EcoRI/BsmBI sites of the pDsRed-ZHX3 (HD2-STOP) to produce pDsRed-ZHX3 (1663-STOP). The EcoRI/BamHI fragment of the resultant plasmid was subcloned into the EcoRI/BamHI sites of the pEGFP-C1E1 to produce pGFP-ZHX3 (555-956).

HisCMCS1 (SEQ ID NO: 32) and H is CMCS2 oligonucleotides (SEQ ID NO: 33) were annealed, phosphorylated and subcloned into the KpnI/EcoRI sites of the pcDNA3.1His-C to give the pcDNA3.1His-C2. The pGST-ZHX1 (272-432) was described previously. A 480-bp BamHI fragment of the pGST-ZHX1 (272-432) was subcloned into the BamHI site of the pcDNA3.1His-C2 to produce pCMV-ZHX1 (272-432).

The nucleotide sequences of all plasmids were confirmed using a DNA sequencer 3100 (Applied Biosystems).

### Reference Example 2

### Library Screening

pDBD-ZHX1 (1-873) (pGBKT7-ZHX1 (1-873)), which expresses the entire coding sequence of human ZHX1 fused to the DNA-binding domain (DBD) of yeast transcription factor GAL4, and the construction of rat granulosa cell and liver cDNA libraries were described previously (Hirano, S., Yamada, K., Kawata, H., Shou, Z., Mizutani, T., Yazawa, T., Kajitani, T., Sekiguchi, T., Yoshino, M., Shigematsu, Y., Mayumi, M., and Miyamoto, K. (2002) Gene 290, 107-114 and others). AH109 yeast cells were transformed with the pDBD-ZHX1 (1-873) plasmid. The strain was used as a bait to screen cDNA libraries. A Tris/EDTA/lithium acetate-based high-efficiency transformation method was used for library screening (Yamada, K., Wang, J.-C., Osawa, H., Scott, D. K., and Granner, D. K. (1998) BioTechniques 24, 596-600). We plated approx. 1.5×10⁷ and 1.1×10⁷ independent clones of the liver and granulosa cell cDNA libraries on to histidine-, tryptophan-, leucine- and adenine-free synthetic dextrose plates supplemented with 4 mM 3-aminotriazole and X-α-gal, respectively. Thus 33 and 109 positive clones were obtained from the primary transformants, respectively. The yeast strain SFY526, which contains a quantifiable lacZ reporter, and either the pGBKT7 or pDBD-ZHX1 (1-873) plasmids, was transformed with plasmids isolated from positive clones in primary screening of the parent vector, pACT2. In the second screening, 16 and 25 clones from liver and granulosa cell cDNA libraries, respectively, specifically exhibited reproducible high β-galactosidase activity. Quantitative β-galactosidase assays, using o-nitrophenyl-β-D-galactoside, were carried out on permeabilized cells, as described previously (Yamada, K., Osawa, H., and Granner, D. K. (1999) FEBS Lett. 460, 41-45; Yamada, K., Printz, R. L., Osawa, H., and Granner, D. K. (1999) Biochem. Biophys. Res. Commun. 261, 614-621 and others). Nucleotide sequences from each positive clone were compared with those entered in the GenBank database using the BLAST sequence search and comparison program.

### Reference Example 3

### Rapid Amplification of cDNA Ends (RACE)

To obtain the 5′ end of the human ZHX3 cDNA, we employed a 5′-RACE method using human testis marathon-ready cDNA and the Advantage 2 PCR kit. Two gene-specific primers, hZHX3-5RACE-As1 (SEQ ID NO: 34), and hZHX3-5RACE-As2 (SEQ ID NO: 35), were used. The 5′-RACE procedure was carried out according to the manufacturer's recommended protocol. Amplified DNA fragments were subcloned into the pGEM-T Easy vector and their nucleotide sequences determined.

### Reference Example 4

### Poly(A)+ RNA Blot Analysis

Human Multiple Tissue Northern Blot and Blot II were hybridized with 0.6-kb EcoR I fragment of the human ZHX3 cDNA, isolated from the pGEM-T Easy hZHX3N plasmid and labelled with [□-³²P] dCTP using BcaBest DNA labelling kit. The ExpressHyb hybridization solution was used for prehybridization and hybridization. Prehybridization, hybridization and washing procedures were performed according to the protocol provided by the supplier.

### Reference Example 5

### Yeast Two-Hybrid System and Liquid β-Galactosidase Assays

To analyse the heterodimerization domain of ZHX1 with ZHX3, SFY526 yeast strains harbouring pDBD or pDBD-G23 were transformed with various truncated forms of ZHX1 fused to the GAL4 AD, or pACT2. To map the heterodimerization domain of ZHX3 with ZHX1 or the homodimerization domain of ZHX3, SFY526 yeast strains harbouring the pDBD, pDBD-ZHX1 (1-873), or pDBD-G23 were transformed with various truncated forms of ZHX3 fused to the GAL4 AD, or pACT2. In order to examine the interaction domain of ZHX3 with NF-YA, SFY526 yeast strains harbouring pDBD or pYA1-269 were transformed with various truncated forms of ZHX3 fused to the GAL4 AD. For mapping the interacting domain of NF-YA with ZHX3, a SFY526 yeast strain harbouring pAD-ZHX3 (242-488) was transformed with various truncated forms of NF-YA fused to the GAL4 DBD.

These β-galactosidase activities were determined as described previously (Yamada, K., Osawa, H., and Granner, D. K. (1999) FEBS Lett. 460, 41-45; Yamada, K., Printz, R. L., Osawa, H., and Granner, D. K. (1999) Biochem. Biophys. Res. Commun. 261, 614-621; Hirano, S., Yamada, K., Kawata, H., Shou, Z., Mizutani, T., Yazawa, T., Kajitani, T., Sekiguchi, T., Yoshino, M., Shigematsu, Y., Mayumi, M., and Miyamoto, K. (2002) Gene 290, 107-114).

### Reference Example 6

### GST Pull-Down Assays

The pGST-ZHX1 (1-873) plasmids have been described previously (Hirano, S., Yamada, K., Kawata, H., Shou, Z., Mizutani, T., Yazawa, T., Kajitani, T., Sekiguchi, T., Yoshino, M., Shigematsu, Y, Mayumi, M., and Miyamoto, K. (2002) Gene 290, 107-114). TOPP3 cells were transformed with pGEX-5X-1, pGST-ZHX1 (1-873), pGST-G23 or the pGST-ZHX3 (1-956) fusion-protein expression plasmid. The preparation of the GST fusion protein, ³⁵S-labelling of in vitro-translated ZHX1 and pull-down analysis have been described previously (Yamada, K., Printz, R. L., Osawa, H., and Granner, D. K. (1999) Biochem. Biophys. Res. Commun. 261, 614-621; Hirano, S., Yamada, K., Kawata, H., Shou, Z., Mizutani, T., Yazawa, T., Kajitani, T., Sekiguchi, T., Yoshino, M., Shigematsu, Y, Mayumi, M., and Miyamoto, K. (2002) Gene 290, 107-114). The pDBD-ZHX3 (1-956) plasmid was employed for the preparation of in vitro-translated ³⁵S-labelled ZHX3. Finally, the beads were resuspended in an equal volume of 2×SDS sample buffer and each supernatant was loaded on to an SDS/PAGE gel (10%), along with a prestained molecular-mass marker. The gel was dried and exposed to a FUJIX imaging plate (Kanagawa, Japan). Interaction signals were detected using the FUJIX BAS-2000 image analysing system. The relative purity and amounts of each fusion protein were determined by gel-staining with Coomassie Brilliant Blue R-250.

### Reference Example 7

### Cell Culture and DNA Transfections

HEK293 cells were cultured in Dulbecco's modified Eagle's medium supplemented with 10% fetal bovine serum at 37° C. in a 5% CO₂ incubator.

DNA transfections were carried out using the Lipofectamine Plus reagents. All plasmids used for the transfection were prepared using a Qiagen plasmid kit, followed by CsC1 gradient ultracentrifugation. Cells (5×10⁴/well) were inoculated in a 24-well plate on the day prior to transfection. The 5xGAL4-pGL3 Control has been described previously (Tanaka, T., Inazu. T., Yamada, K., Myint, Z., Keng, V. W., Inoue, Y., Taniguchi, N., and Noguchi, T. (1999) Biochem. J. 339, 111-117). 5xGAL4-pGL3 Control or pGL3-Control was employed as the reporter plasmid. For the determination of transcriptional activity of ZHX3, 100 ng of a reporter plasmid, 2 ng of the pRL-CMV and the indicated amount of GAL4 DBD-ZHX3 fusion-protein expression plasmid were transfected. The total amount of plasmid (152 ng) was adjusted by the addition of the pSG424, if required. For the analysis of effects of heterodimerization of ZHX3 with ZHX1 on the transcriptional activity of ZHX3, 100 ng of a reporter plasmid, 2 ng of the pRL-CMV, 50 ng of GAL4 DBD fusion-protein expression plasmids, and the indicated amount of the expression plasmid for the dimerization domain of the human ZHX1, pCMV-ZHX1 (272-432), were transfected. The total amount of plasmid (202 ng) was adjusted by the addition of the pcDNA3.1His-C2, if necessary. For observation of the green fluorescent protein (GFP) fusion protein, 300 ng of the indicated GFP plasmid was transfected. Then, 3 h after transfection, the medium was changed. After 48 h the cells were subjected to luciferase assays or observed with a laser microscope (Olympus). Firefly and sea pansy luciferase assays were performed according to the manufacturer's recommended protocol (Promega). Luciferase activities were determined by a Berthold Lumat model LB 9501 (Wildbad, Germany). Firefly luciferase activities (relative light units) were normalized by sea pansy luciferase activities.

### Example 1

In this example, to analyze the molecular mechanism of transcriptional repression by ZHX1 and to examine the issue of whether the human ZHX1 interacts with either a known or a novel transcription factor, the inventors conducted a screening of ZHX1-interacting proteins. An entire coding sequence of the human ZHX1 was fused to the GAL4 DBD and this chimaeric protein was employed as the bait to screen rat liver and granulosa cell cDNA libraries (reference example 2) using the yeast two-hybrid system (reference example 5). Approx. 1.5×10⁷ and 1.1×10⁷ independent clones of each library were screened, and 16 and 25 clones showed reproducible His⁺, Ade⁺ and α-gal-positive properties, respectively. The inventors isolated plasmids that encode the GAL4 AD fusion protein from these clones. After determination of their nucleotide sequences, they were compared with the GenBank database using the BLAST search program. The results were shown in Table 1.

TABLE 1
The ZHX1-interacting proteins

| Protein | Number of clone |
|---|---|
| BS69 corepressor | 9 |
| Nuclear protein, ataxia-telangiectasia-like protein | 5 |
| Androgen-induced alsose reductase | 3 |
| ATF-IP | 2 |
| Spinocerebellar ataxia type I | 2 |
| Zyxin | 2 |
| Elf-1 | 1 |
| Eleven-nineteen lysine-rich leukemia gene | 1 |
| ZHX1 | 8 |
| unknown | 8 |

As shown in Table 1, the BS69 co-repressor, nuclear protein, ataxia-telangiectasia-like protein, androgen-induced aldose reductase, activating transcription factor (ATF)-interacting protein (ATF-IP), spinocerebellar ataxia type I, zyxin, Elf-1, eleven-nineteen lysine-rich leukemia gene and ZHX1 were cloned as known proteins (Hirano, S., Yamada, K., Kawata, H., Shou, Z., Mizutani, T., Yazawa, T., Kajitani, T., Sekiguchi, T., Yoshino, M., Shigematsu, Y., Mayumi, M., and Miyamoto, K. (2002) Gene 290, 107-114 and others). Eight clones encoded unknown proteins. Interestingly, three clones, G23, G58 and L26, encoded both Znf and HD motifs. A detailed nucleotide sequence analysis showed that the nucleotide sequence of G23 was included in that of G58, and that the nucleotide sequence exhibited a similarity to that of partially cloned human KIAA0395 cDNA. The inventors focused on the analysis of these clones in this study. The L26 clone, differing from the KIAA0395, has been denoted ZHX2 and will be reported on in the future.

In order to isolate the 5′-non-coding sequence and the remaining coding region of the human KIAA0395 cDNA, the inventors then employed a 5′-RACE method (reference example 3). Using combinations of gene-specific primers and adaptor primers, a cDNA fragment was obtained by PCR using human testis marathon cDNA as a template. Finally, the size of the full-length cDNA was determined to be 9302 bp.

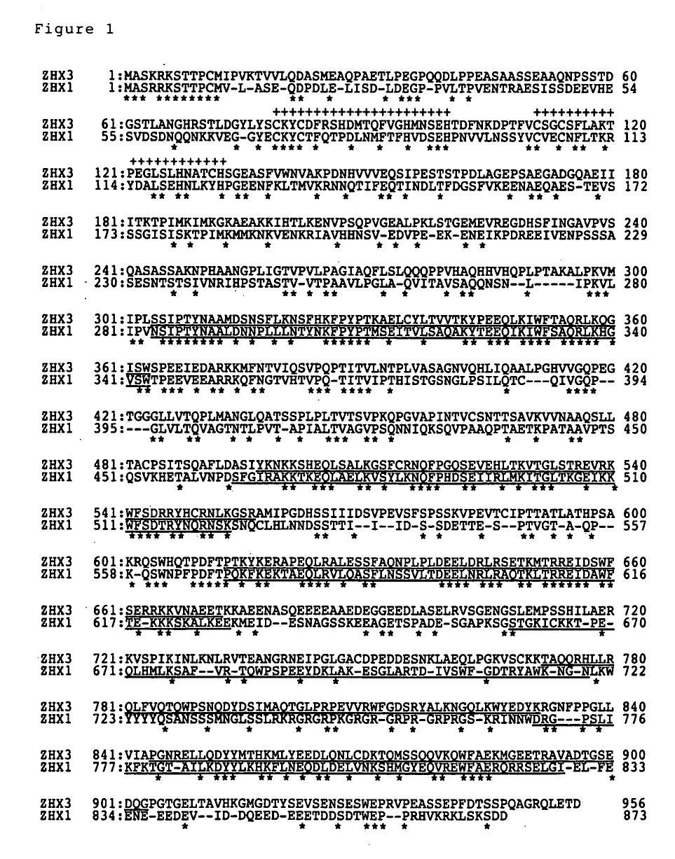

Very interestingly, the full-length cDNA has an open reading frame of 956 amino acid residues and the deduced amino acid sequence of the protein contains two Cys₂-His₂-type Znf motifs and five HDs as well as ZHX1. The amino acid sequence is shown in SEQ ID NO: 1. Hereafter, the inventors refer to the protein as ZHX3. The name ZHX3 has been submitted to the HUGO Nomenclature Committee with the name zinc-fingers and homeoboxes 3. FIG. 1 shows a comparison of deduced amino acid sequences of human ZHX3 with human ZHX1

The human ZHX3 protein has a predicted molecular mass of 104.7 kDa and a pI of 5.68. Whereas pAD-G58 and pAD-G23 encoded amino acid sequence between 114 and 642 and between 242 and 615 of the ZHX3, respectively, KIAA0395 encoded amino acid sequence between 498 and 956 of the human ZHX3. A glutamic-acid-rich region that may act as a transcription regulatory domain existed in the amino acid sequence between 670 and 710 and no putative nuclear-localization signals exist. The similarity in nucleotide sequences in the coding region and amino acid sequences between ZHX3 and ZHX1 were 46.9% and 34.4%, respectively.

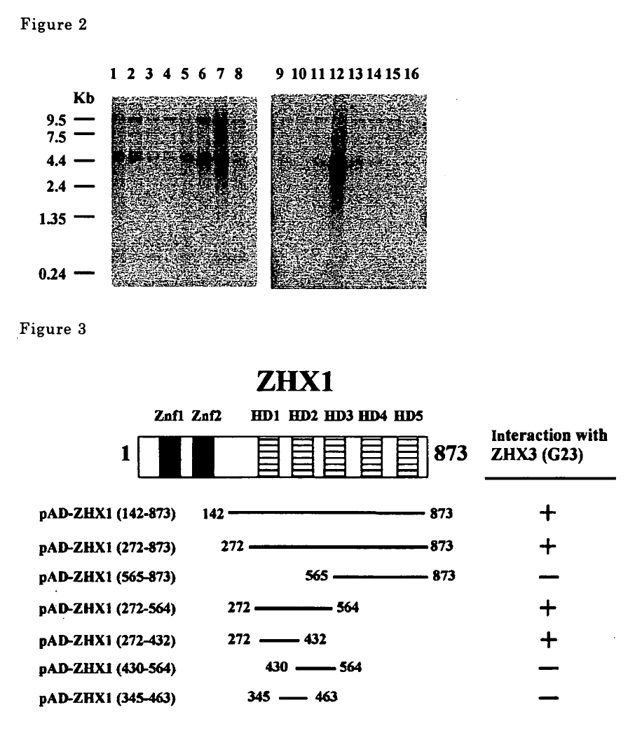

Then, the tissue distribution of human ZHX3 mRNA was determined by Northern blot analysis (reference example 4). As shown in FIG. 2, human ZHX3 mRNA was detected as multiple bands, 9.4, 7.3, 5.0 and 4.6 kb in length. Since the size of the inventor's cloned insert was 9302 bp, it is almost identical with the full-length transcript. These transcripts were observed in all the tissues examined, although the intensity varied among tissues. This indicates that human ZHX3 mRNA is expressed ubiquitously.

### Example 2

To examine the issue of which domain of ZHX1 is required for interaction with ZHX3 in this example, the inventors conducted mapping of the minimal hetero-dimerization domain between ZHX1 and ZHX3. A yeast strain SFY526 was transformed with the pDBD, which expresses GAL4 DBD alone, or pDBD-G23, which encodes amino acid residues 242-615 of the ZHX3 fused to GAL4 DBD. The two yeast strains were used as the reporter yeasts. pACT2, which expresses GAL4 AD alone or some plasmids, which encode various truncated forms of ZHX1 fused to the GAL4 AD, were employed as the prey plasmids.

When a reporter yeast harbouring the pDBD was transformed with these prey plasmids, they showed low β-galactosidase activities (results not shown). In addition, when a reporter yeast harbouring the pDBD-G23 was transformed with the pACT2, pAD-ZHX1 (565-873), pAD-ZHX1 (430-564) or pAD-ZHX1 (345-463), these yeasts also showed low β-galactosidase activities (FIG. 3). In contrast, when the yeast was transformed with the pAD-ZHX1 (142-873), pAD-ZHX1 (272-873), pAD-ZHX1 (272-564) or pAD-ZHX1 (272-432), high β-galactosidase activities were observed. pAD-ZHX1 (272-432) encodes amino acid residues between 272 and 432 of ZHX1.

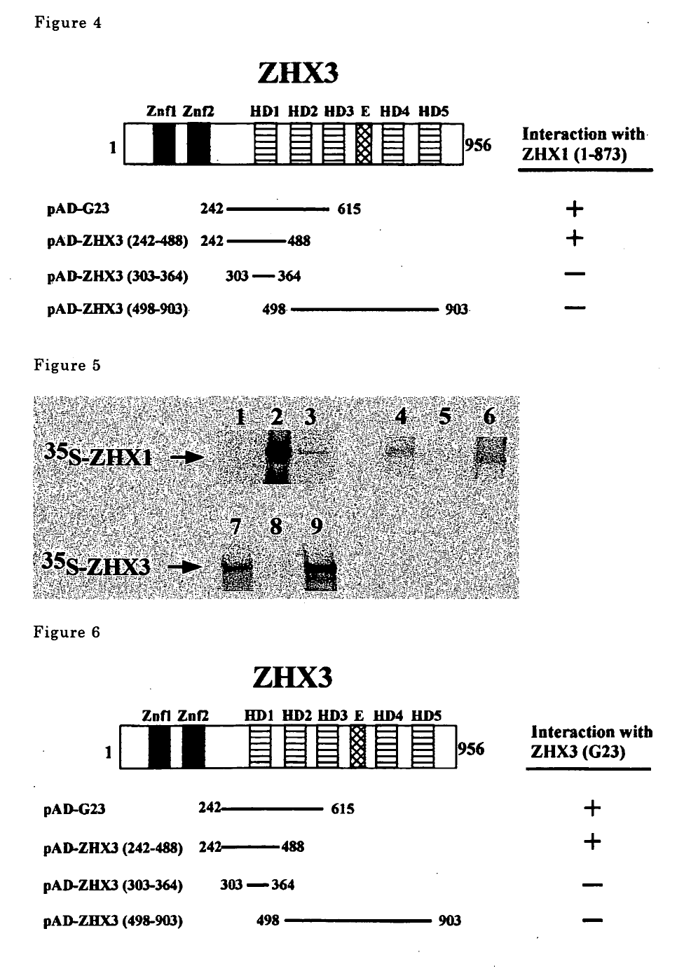

The inventors then determined the issue of which domain of ZHX3 is required for an interaction with ZHX1. Yeast strain SFY526 was transformed with pDBD-ZHX1 (1-873), which encodes an entire coding sequence of the ZHX1 fused to GAL4 DBD, or pDBD. The two yeast strains were used as the reporter yeasts. The pACT2 or some plasmids encoding various truncated forms of ZHX3 fused to the GAL4 AD were employed as prey plasmids (FIG. 4). When a reporter yeast harbouring the pDBD was transformed with these prey plasmids, they showed low β-galactosidase activities (results not shown). These yeasts showed high β-galactosidase activities only when a reporter yeast harbouring pDBD-ZHX1 (1-873) was transformed with pAD-G23 or pAD-ZHX3 (242-488). pAD-ZHX3 (242-488) encodes the amino acid residues between 242 and 488 of ZHX3.

These results reveal that ZHX1 and ZHX3 form a heterodimer via each region including HD1. The inventors also carried out in vitro GST pull-down assays (reference example 6) to verify the specific interaction between ZHX1 and ZHX3. The inventors employed four plasmids, pGEX-5X-1, which expresses GST alone, pGST-G23, which encodes the amino acids 242-615 of human ZHX3 fused to GST, pGST-ZHX3 (1-956), which expresses the entire coding region of human ZHX3 protein fused to GST, and pGST-ZHX1 (1-873), which expresses the entire coding region of human ZHX1 protein fused to GST. These proteins were expressed in Escherichia coli and immobilized on to glutathione-Sepharose beads. The in vitro-translated, ³⁵S-labelled full-length of human ZHX1 was found to bind to GST-G23 and GST-ZHX3 (1-956) but not to GST alone (FIG. 5, lanes 3 and 6). In addition, the in vitro-translated, 35S-labelled full-length human ZHX3 was found to bind to GST-ZHX1 (1-873) but not to GST alone (FIG. 5, lane 9). In contrast, an unprogrammed reticulocyte lysate failed to bind to any of the proteins (results not shown).

These results indicate that ZHX1 is able to form a heterodimer with ZHX3 both in vivo and in vitro.

### Example 3

Since ZHX1 forms a homodimer, the inventors conducted a mapping of the minimal homodimerization domain of ZHX3, to investigate formation of a homodimer for ZHX3 using the yeast two-hybrid system (reference example 5). Two SFY526 yeast strains harbouring pDBD or pDBD-G23 were used as the reporter yeasts. The inventors prepared various prey plasmids, pACT2, pAD-G23, pAD-ZHX3 (242-488), pAD-ZHX3 (303-364) and pAD-ZHX3 (498-903). These plasmids were transformed into the reporter yeasts and β-galactosidase activity was determined in each case (FIG. 6). When the reporter yeast harbouring pDBD was transformed with the plasmids, they showed very low β-galactosidase activities (results not shown). In addition, when a reporter yeast harbouring pDBD-G23 was transformed with pACT2, pAD-ZHX3 (303-364) and pAD-ZHX3 (498-903), very low β-galactosidase activity was also detected. In contrast, the yeast transformed with pAD-G23 or pAD-ZHX3 (242-488) expressed high β-galactosidase activities. These results indicate that ZHX3 is able to form a homodimer via the region between residues 242 and 488.

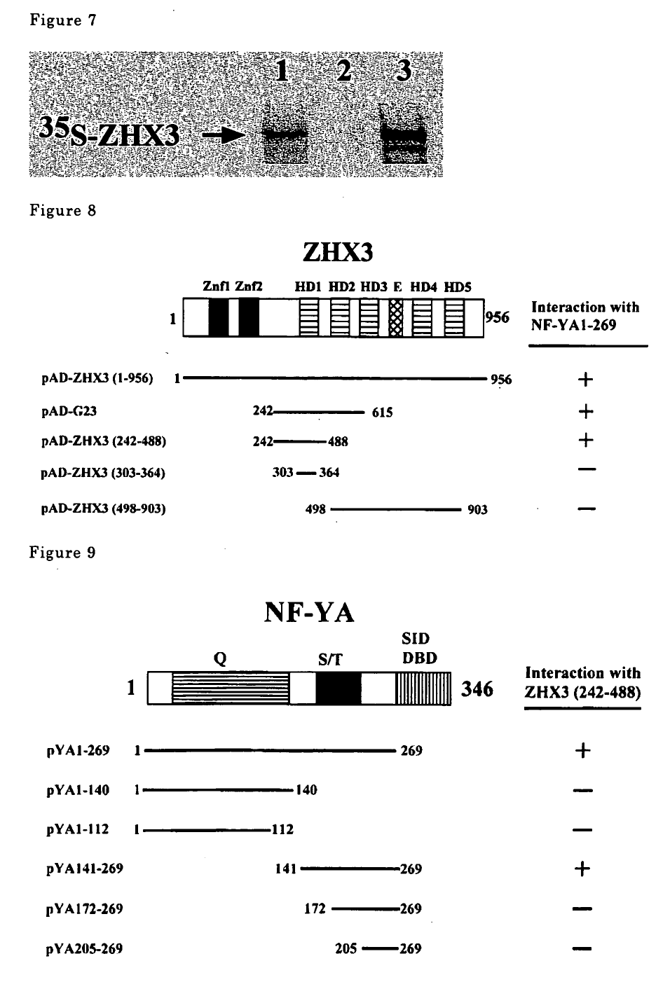

The inventors then carried out in vitro GST pull-down assays to verify the homodimerization of ZHX3. The inventors employed two plasmids, pGEX-5X-1 and pGST-ZHX3 (1-956). These proteins were expressed in E. coli and immobilized on to glutathione-Sepharose beads. The in vitro-translated, ³⁵S-labelled full-length human ZHX3 was found to bind to GST-ZHX3 (1-956) but not to GST alone (FIG. 7). In contrast, an unprogrammed reticulocyte lysate failed to bind to any of the proteins (results not shown). These results indicate that ZHX3 is able to form a homodimer in vivo and in vitro.

### Example 4

In this example, the inventors investigated the issue whether ZHX3 also interacts with the AD of the NF-YA. Human ZHX1 was originally cloned as a protein that interacts with NF-YA (Yamada, K., Printz, R. L., Osawa, H., and Granner, D. K. (1999) Biochem. Biophys. Res. Commun. 261, 614-621). The inventors examined the interaction of ZHX3 with NF-YA using the yeast two-hybrid system (reference example 5). The inventors used two reporter-yeast strains, which are transformed with pDBD or pYA 1-269. pYA 1-269 expresses the AD of the NF-YA fused to the GAL4 DBD. pACT2, pAD-ZHX3 (1-956), pAD-G23, pAD-ZHX3 (242488), pAD-ZHX3 (303-364) and pAD-ZHX3 (498-903) were transformed into the reporter yeast strains and their β-galactosidase activities determined.

When a reporter yeast harbouring the pDBD was transformed with these plasmids, their β-galactosidase activities were found to be quite low (results not shown). As shown in the middle of FIG. 8, when a reporter yeast harbouring pYA1-269 was transformed with pACT2, pAD-ZHX3 (303-364) or pAD-ZHX3 (498-903), their β-galactosidase activities were also low. However, when the yeast was transformed with pAD-ZHX3 (1-956), pAD-G23 or pAD-ZHX3 (242-488), high levels of β-galactosidase activity were detected. These results indicate that ZHX3 interacts with the AD of the NF-YA, and that the amino acid sequence between residues 242 and 488 is essential for this interaction.

The inventors next identified the minimal interaction domain of NF-YA with ZHX3 using the yeast two-hybrid system (reference example 5). As shown in FIG. 9, the AD of NF-YA consists of a glutamine-rich and a serine/threonine-rich domain. The SFY526 yeast strain harbouring pAD-ZHX3 (242-488) was used as a reporter yeast. Various plasmids expressing truncated forms of the NF-YA fused to the GAL4 DBD were transformed in the yeast and their β-galactosidase activities determined. As a result, only yeasts harbouring the pYA 1-269 or pYA 141-269, both of which contain amino acids 141-269 of NF-YA, showed a high level of β-galactosidase activity. These results indicate that a serine/threonine-rich AD of NF-YA represents the minimal interaction domain with ZHX3.

### Example 5

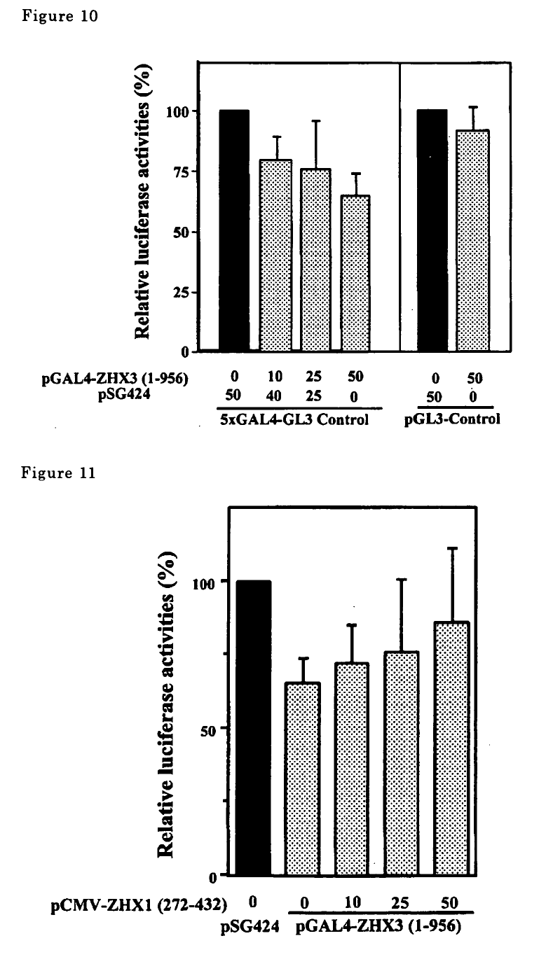

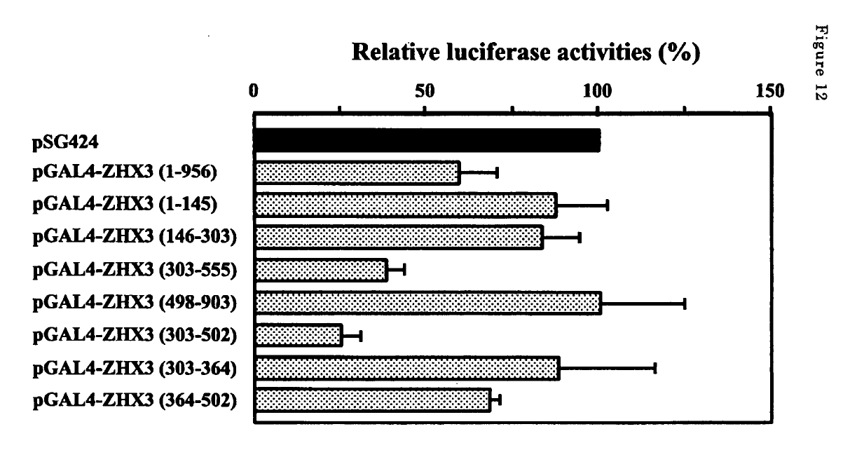

In this example, to confirm that ZHX3 is a transcriptional repressor, the inventors determined the transcriptional role of ZHX3 using a mammalian one-hybrid system (reference example 7). The 5xGAL4-pGL3 Control plasmid, in which five copies of the GAL4-binding site had been inserted upstream of the simian virus 40 (SV40) promoter of pGL3-Control, was employed as a reporter plasmid. Two effector plasmids, pSG424, which expresses GAL4 DBD alone, and pGAL4-ZHX3 (1-956), which expresses the entire coding region of human ZHX3 fused to the C-terminus of the GAL4 DBD, were prepared. As shown in FIGS. 10 and 11, when 5xGAL4-pGL3 Control and various amounts of pGAL4-ZHX3 (1-956) were co-transfected into HEK293 cells, the luciferase activity was decreased in a dose-dependent manner. The maximal inhibition was obtained with 50 ng of the pGAL4-ZHX1 (1-956). In contrast, when the pGL3-Control lacking five copies of the GAL4-binding sites was transfected with the pSG424 or pGAL4-ZHX3 (1-956), the luciferase activities remained unchanged (FIG. 10). These results show that the GAL4-ZHX3 fusion protein decreases luciferase activity in a GAL4-binding-site-dependent manner, indicating that ZHX3 acts as a transcriptional repressor.

The inventors then examined the issue of whether heterodimerization of ZHX3 with ZHX1 is required for its transcriptional repressor activity. The inventors prepared the pCMV-ZHX1 (272-432), in which amino acids 272-423 of ZHX1 are expressed. Although this region corresponds to the dimerization domain of ZHX1 with ZHX3, it does not contain the repressor domain of ZHX1 (Yamada, K., Kawata, H., Matsuura, K., Shou, Z., Hirano, S., Mizutani, T., Yazawa, T., Yoshino, M., Sekiguchi, T., Kajitani, T., and Miyamoto. K. (2002) Biochem. Biophys. Res. Comm. 279, 368-374). Therefore, the overexpression of this protein functions as a dominant-negative form of ZHX1. When the plasmid was co-transfected in the above assay system, the luciferase activity was increased in a dose-dependent manner (FIG. 11). In contrast, co-transfection of the pcDNA3.1H is C-2 had no effect on luciferase activity. These results suggest that heterodimerization of ZHX3 with ZHX1 is a prerequisite for repressor activity.

Finally, to determine the minimal repressor domain of ZHX3, 5xGAL4-pGL3 Control was transfected with various plasmids, pGAL4-ZHX3 (1-145), pGAL4-ZHX3 (146-303), pGAL4-ZHX3 (303-555) or pGAL4-ZHX3 (498-903) (FIG. 12). Only pGAL4-ZHX3 (303-555), which expresses amino acids 303-555, led to a decrease in luciferase activity.

For a more detailed analysis, the inventors prepared the effector plasmids pGAL4-ZHX3 (303-502), pGAL4-ZHX3 (303-364) and pGAL4-ZHX3 (364-502). Only when pGAL4-ZHX3 (303-502) was transfected with the reporter plasmid did the luciferase activity decrease (FIG. 12). These results show that the amino acid sequence between residues 303 and 502 of ZHX3 are essential for repressor activity.

### Example 6

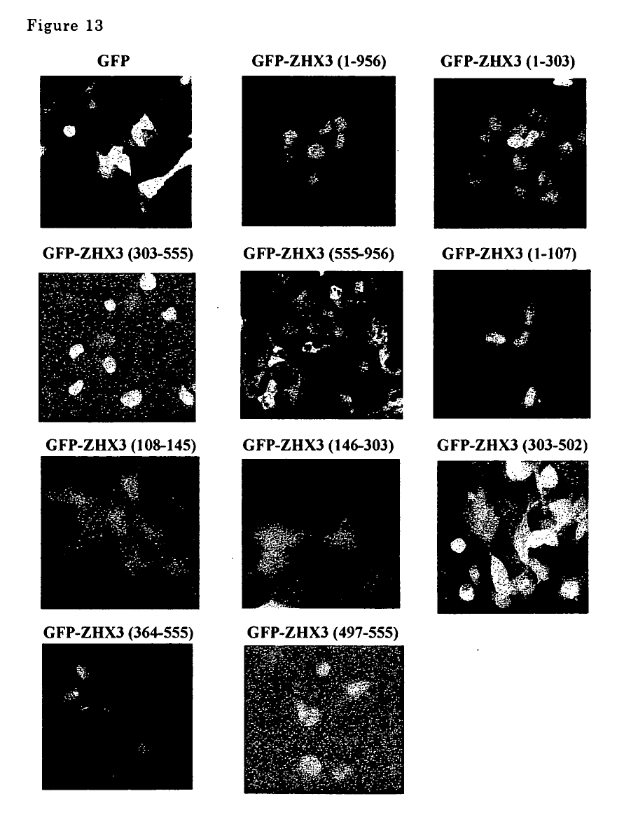

To examine the subcellular localization of the ZHX3 protein, the inventors determined the subcellular localization of ZHX3 and mapped NLSs, employing the GFP-ZHX3 fusion-protein expression system. Various truncated forms of ZHX3 fused to GFP were prepared. These plasmids were transfected into HEK293 cells and the subcellular localization of the GFP fusion proteins observed. pEGFP-C1E1, encoding GFP protein alone, was observed in the whole cell (FIG. 13). In contrast, GFP-ZHX3 (1-956), in which full-length ZHX3 was fused to the C-terminus of GFP, was localized in the nuclei. To determine the NLS of ZHX3, various plasmids were transfected. When pGFP-ZHX3 (1-303), pGFP-ZHX3 (303-555) and pGFP-ZHX3 (555-956) were transfected, both pGFP-ZHX3 (1-303) and pGFP-ZHX3 (303-555) were located in the nuclei. In contrast, when pGFP-ZHX3 (555-956) was transfected, the protein was localized outside of the nuclei. These results suggest that ZHX3 contains two NLS and a nuclear export signal (NES). To map the minimal NLSs, various plasmids were constructed. Only when three plasmids, pGFP-ZHX3 (1-107), pGFP-ZHX3 (364-555) and pGFP-ZHX3 (497-555), were transfected, the nuclear localization of ZHX3 was observed. These results show that ZHX3 is able to localize in the nuclei as a GFP fusion protein, and that two NLSs of ZHX3 are located in amino acids 1-107 and 497-555.

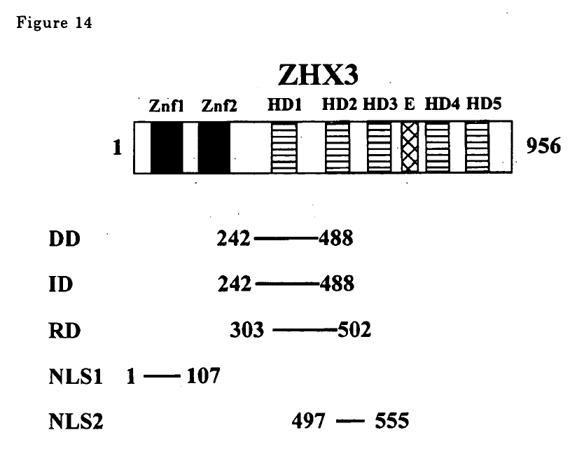

In the above examples, the inventors conducted a search for ZHX1-interacting proteins, mainly by analysing a novel transcriptional repressor of them, ZHX3, and mapped its functional domains. The minimal functional domains of ZHX3 are summarized in FIG. 14. ZHX3 as well as ZHX1 contains two Znf motifs and five HDs, forms a homodimer, interacts with the AD of NF-YA, and is localized in the nucleus. In addition, ZHX3 mRNA is expressed ubiquitously. From these findings, the inventors conclude that both ZHX1 and ZHX3 are members of the same family, namely the ZHX family.

While the similarity of the entire amino acid sequences of ZHX1 and ZHX3 was 34.4%, the two Znf motifs and five HDs were highly conserved. Similarities in the amino acid sequences of Znf1, Znf2, HD1, HD2, HD3, HD4 and HD5 between ZHX1 and ZHX3 were 50.0, 45.5, 61.7, 50.0, 53.3, 43.3 and 33.3%, respectively. The HD4 showed a much lower similarity than the other domains. A unique glutamic-acid-rich acidic region is located in the amino acid sequence between residues 670 and 710 of ZHX3 (FIGS. 1 and 9). Generally, the Znf motif, the HD and the acidic region are responsible for the functional properties of the transcription factor. For example, both the Znf motif and HD, which consist of 60 amino acids, are required for binding to the cognate DNA sequence, the glutamic-acid-rich regions are involved in transcriptional activity, and the basic region is the DBD or NLS (Gehring, W. J., Affolter, M., and Burglin, T. (1994) Annu. Rev. Biochem. 63, 487-526 and others).

ZHX3 not only forms a heterodimer with ZHX1 but also forms a homodimer. Amino acids 242-488 of ZHX3 are necessary and sufficient for these dimerizations (FIGS. 3-7). A minimal domain for the homo- and hetero-dimerization of ZHX1 with ZHX3 was mapped to amino acids 272-432 of ZHX1 (FIGS. 3-5). These regions include the HD1 but HD1 alone failed to dimerize (FIGS. 3-9). A more extensive region including HD1 is required for dimerization. Furthermore, both ZHX3 and ZHX1 interact with the AD of NF-YA; the former interacts with a serine/threonine-rich AD and the latter with a glutamine-rich AD of the NF-YA, respectively (FIGS. 8 and 9). An interaction domain of ZHX3 with the AD of NF-YA was mapped to the same region as the dimerization domain of ZHX3 (FIGS. 8 and 9). In contrast, the amino acid sequence between 272 and 564, which contains the HD1-HD2 region of human ZHX1, is required for its interaction (Yamada, K., Osawa, H., and Granner, D. K. (1999) FEBS Lett. 460, 41-45). The issue of whether a heterodimer complex of ZHX1 with ZHX3 interacts with different ADs of NF-YA remains to be determined.

ZHX3 is a transcriptional repressor (FIGS. 10-12). The minimal repressor domain of ZHX3 is mapped to an overlapping region with both dimerization and interaction domains. Interestingly, the overexpression of the heterodimerization domain of ZHX1, which is not responsible for repressor activity, led to a decrease in the repressor activity of ZHX3 (FIG. 11). This raises the possibility that ZHX3 itself has no repressor activity and that the observed activity is dependent upon the repressor activity of a dimerization partner, ZHX1, in which the repressor domain is located in the C-terminus of the acidic region (Yamada, K., Kawata, H., Matsuura, K., Shou, Z., Hirano, S., Mizutani, T., Yazawa, T., Yoshino, M., Sekiguchi, T., Kajitani, T., and Miyamoto. K. (2002) Biochem. Biophys. Res. Comm. 279, 368-374). However, it cannot be ruled out that the dimerization domain of ZHX3 has bona fide repressor activity. It is unclear whether ZHX3 is a DNA-binding protein or not. As a result, it appears that a region of ZHX3 including the HD1 region is a pleiotropic domain; a homo- and hetero-dimerization domain with ZHX1, an interaction domain with the AD of NF-YA, and a repressor domain.

Regions of transcriptional regulation interact with cofactors to function as transcriptional repressors (Hu, X., and Lazar, M. A. (2000) Trends Endocrinol. Metab. 11, 6-10 and others). These cofactors include mSin3A/B, histone deacetylases and the nuclear co-repressor (N-CoR)/silencing mediator of receptor transcription. As ZHX1-interacting proteins the inventors cloned two co-repressors, BS69 and ATF-IP (Table 1) (Hateboer, G., Gennissen, A., Ramos, Y. F. M., Kerkhoven, R. M., Sonntag-Buck, V., Stunnenberg, H. G., and Bernards. R. (1995) EMBO J. 14, 3159-3169 and others). BS69 was first identified as a protein that interacts directly with the AD of the 289R adenovirus type 5 E1A protein. It has been also reported that BS69 mediates repression, at least in part, through an interaction of the MYND domain of BS69 with the co-repressor N-CoR (Masselink, H., and Bernards, R. (2000) Oncogene 19, 1538-1546). In contrast, ATF-IP interacts with several components of the basal transcription machinery (TFIIE and TFIIH), including RNA polymerase II holoenzyme (DeGraeve, F., Bahr, A., Chatton, B., and Kedinger, C. (2000) Oncogene 10, 1807-1819). When ZHX1 and ZHX3 act as a transcriptional repressor, it could interact with these co-repressors, thus repressing gene transcription.

Furthermore, both ZHX1 and ZHX3 are NF-YA-interacting proteins. It has been reported that NF-Y is associated with co-activators, p300 and p300/cAMP response element-binding protein-binding protein-associated factor (P/CAF) (Mantovani, R. (1999) Gene 239, 15-27). In particular, P/CAF with histone acetyltransferase activity interacts with the NF-YA to form a transcriptionally active NF-Y complex (Mantovani, R. (1999) Gene 239, 15-27). Therefore, it is likely that combinations of interactions among ZHX1ZHX3 and NF-YA affect the transcriptional activity of NF-Y For example, either ZHX1 or ZHX3, or both, may enhance or interfere with the association of P/CAF with the NF-Y, thus regulating NF-Y activity. In addition, the transcriptional repressor, a member of the ZHX family, and a co-repressor are able to directly associate with the NF-YA, thus inhibiting NF-Y activity. In any case, it is possible that the ZHX proteins participate in the regulation of a number of NF-Y-regulatable genes.

Although ZHX3 mRNA is expressed ubiquitously, it was found to be expressed more highly in skeletal muscle, kidney and testis (FIG. 2). The size of ZHX3 mRNA varied as determined by Northern blot analysis. The cloned insert contains 9302 bp and the size is the same as the largest transcript of ZHX3. When a search for the human ZHX3 gene was conducted using the database compiled by the Human Genome Project, it was found to be located in chromosome 20q. This suggests that the ZHX3 gene exists as a single copy per haploid human genome. The nucleotide sequence of ZHX3 cDNA revealed that multiple polyadenylation signals exist in the 3′-non-coding region. Therefore, it is likely that smaller mRNAs might be produced by the use of different polyadenylation signals from a single gene rather than by the existence of other ZHX3-related mRNAs.

When the entire coding region of ZHX3 was fused to the C-terminal end of the GFP, it became localized in the nuclei (FIG. 13). There were two NLSs of ZHX3, amino acids 1-107 and 497-555. On the other hand, amino acids 498-956 of ZHX3 fused to GFP become exclusively localized not in the entire cell but external to the nucleus. GFP alone or the GFP-ZHX1 fusion protein lacking the NLS was localized to entire cells (FIG. 13). This indicates that this region of ZHX3 contains a nuclear-export signal. Therefore, ZHX3 is a more complicated protein that contains two NLSs and a nuclear-export signal in one molecule. In many other proteins, including ZHX1, it has been reported that the NLS was mapped to a cluster of basic amino acid residues (Yamada, K., Kawata, H., Matsuura, K., Shou, Z., Hirano, S., Mizutani, T., Yazawa, T., Yoshino, M., Sekiguchi, T., Kajitani, T., and Miyamoto. K. (2002) Biochem. Biophys. Res. Comm. 279, 368-374 and others). This region is associated with nuclear-importing proteins such as importin a and is then translocated from the cytoplasm to the nuclei (Kaffman, A., and O'Shea, E. K. (1999) Annu. Rev. Cell Dev. Biol. 15, 291-339). However, ZHX3 may associate with other molecules in order to be translocated to the nuclei, since the two NLSs of ZHX3 are not located in the basic region and do not exhibit any similarity with previously reported NLS.

1. A drug agent to repress transcription comprising a protein or a peptide of (1) to (4) as an effective component:
(1) a protein or a peptide having an amino acid sequence of SEQ ID NO: 1
(2) a protein or a peptide having an amino acid sequence comprising a deletion, substitution or addition of one or several amino acids with respect to the amino acid sequence of SEQ ID NO: 1, and having a transcriptional repressor activity
(3) a protein or a peptide comprising the functional domain of an amino acid sequence of SEQ ID NO: 1
(4) a protein or a peptide comprising an amino acid sequence comprising a deletion, substitution or addition of one or several amino acids with respect to the functional domain of an amino acid sequence of SEQ ID NO: 1, and having a transcriptional repressor activity.

2. The drug agent of claim 1, wherein said functional domain consists of amino acids 303-502 of SEQ ID NO: 1.

3. (canceled)

4. A therapeutic product for hepatoma to repress transcription of genes expressed specifically in hepatoma cells, comprising a protein or a peptide of (1) to (4) as an effective component:
(1) a protein or a peptide having an amino acid sequence of SEQ ID NO: 1
(2) a protein or a peptide having an amino acid sequence comprising a deletion, substitution or addition of one or several amino acids with respect to the amino acid sequence of SEQ ID NO: 1, and having a transcriptional repressor activity
(3) a protein or a peptide comprising the functional domain of the amino acid sequence of SEQ ID NO: 1
(4) a protein or a peptide comprising an amino acid sequence comprising a deletion, substitution or addition of one or several amino acids with respect to the functional domain of the amino acid sequence of SEQ ID NO: 1, and having a transcriptional repressor activity.

5. The therapeutic product for hepatoma of claim 4, wherein said functional domain consists of amino acids 303-502 of SEQ ID NO: 1.

6. The therapeutic product for hepatoma of claim 4, wherein said genes are type II hexokinase or pyruvate kinase M gene.

7. A screening agent to screen drug agents with transcriptional repressor activity, comprising an antibody specific to a protein or a peptide of (1) to (4) as an effective component:
(1) a protein or a peptide having an amino acid sequence of SEQ ID NO: 1
(2) a protein or a peptide having an amino acid sequence comprising a deletion, substitution or addition of one or several amino acids with respect to the amino acid sequence of SEQ ID NO: 1, and having a transcriptional repressor activity
(3) a protein or a peptide comprising the functional domain of the amino acid sequence of SEQ ID NO: 1
(4) a protein or a peptide comprising an amino acid sequence comprising a deletion, substitution or addition of one or several amino acids with respect to the functional domain of the amino acid sequence of SEQ ID NO: 1, and having a transcriptional repressor activity.

8. The screening agent of claim 7, wherein said functional domain consists of amino acids 303-502 of SEQ ID NO: 1.

9. A protein or a peptide of (3) or (4):
(3) a protein or a peptide comprising the functional domain of the amino acid sequence of SEQ ID NO: 1
(4) a protein or a peptide comprising an amino acid sequence comprising a deletion, substitution or addition of one or several amino acids with respect to the functional domain of the amino acid sequence of SEQ ID NO: 1, and having a transcriptional repressor activity.

10. A protein or a peptide of (3) or (4) to repress transcription of genes expressed specifically in hepatoma cells:
(3) a protein or a peptide comprising the functional domain of the amino acid sequence of SEQ ID NO: 1
(4) a protein or a peptide comprising an amino acid sequence comprising a deletion, substitution or addition of one or several amino acids with respect to the functional domain of the amino acid sequence of SEQ ID NO: 1, and having a transcriptional repressor activity.

11. The protein or a peptide of claim 10, wherein said genes are type II hexokinase or pyruvate kinase M gene.

12. An antibody specific to a protein or a peptide of (3) or (4):
(3) a protein or a peptide comprising the functional domain of the amino acid sequence of SEQ ID NO: 1
(4) a protein or a peptide comprising an amino acid sequence comprising a deletion, substitution or addition of one or several amino acids with respect to the functional domain of the amino acid sequence of SEQ ID NO: 1, and having a transcriptional repressor activity.


SEQUENCE LISTING (36 sequences)

SEQ ID NO: 1 - protein, 956 aa, Homo sapiens
MASKRKSTTPCMIPVKTVVLQDASMEAQPAETLPEGPQQDLPPEASAASSEAAQNPSSTDGSTLANGHRSTLDGYLYSCKYCDFRSHDMTQFVGHMNSEHTDFNKDPTFVCSGCSFLAKTPEGLSLHNATCHSGEASFVWNVAKPDNHVVVEQSIPESTSTPDLAGEPSAEGADGQAEIIITKTPIMKIMKGKAEAKKIHTLKENVPSQPVGEALPKLSTGEMEVREGDHSFINGAVPVSQASASSAKNPHAANGPLIGTVPVLPAGIAQFLSLQQQPPVHAQHHVHQPLPTAKALPKVMIPLSSIPTYNAAMDSNSFLKNSFHKFPYPTKAELCYLTVVTKYPEEQLKIWFTAQRLKQGISWSPEEIEDARKKMFNTVIQSVPQPTITVLNTPLVASAGNVQHLIQAALPGHVVGQPEGTGGGLLVTQPLMANGLQATSSPLPLTVTSVPKQPGVAPINTVCSNTTSAVKVVNAAQSLLTACPSITSQAFLDASIYKNKKSHEQLSALKGSFCRNQFPGQSEVEHLTKVTGLSTREVRKWFSDRRYHCRNLKGSRAMIPGDHSSIIIDSVPEVSFSPSSKVPEVTCIPTTATLATHPSAKRQSWHQTPDFTPTKYKERAPEQLRALESSFAQNPLPLDEELDRLRSETKMTRREIDSWFSERRKKVNAEETKKAEENASQEEEEAAEDEGGEEDLASELRVSGENGSLEMPSSHILAERKVSPIKINLKNLRVTEANGRNEIPGLGACDPEDDESNKLAEQLPGKVSCKKTAQQRHLLRQLFVQTQWPSNQDYDSIMAQTGLPRPEVVRWFGDSRYALKNGQLKWYEDYKRGNFPPGLLVIAPGNRELLQDYYMTHKMLYEEDLQNLCDKTQMSSQQVKQWFAEKMGEETRAVADTGSEDQGPGTGELTAVHKGMGDTYSEVSENSESWEPRVPEASSEPFDTSSPQAGRQLETD

SEQ ID NO: 2 - protein, 522 aa, Rattus norvegicus
CSFLAKTPEGLSLHNAKCHSGEASFLWNVTKPDNHVVVEQSVPENASSSVLAGESTEGTEIIITKTPIMKIMKGKAEAKKIHMLKENAPTQPGGEALPKPLAGETEGKEGDHTFINGATPVSQASANSTKPPHTANGPLIGTVPVLPAGIAQFLSLQQPTVHPQHHPHQPLPTSKALPKVMIPLSSIPTYNAAMDSNSFLKNSFHKFPYPTKAELCYLTVVTKYPEEQLKIWFTAQRLKQGISWSPEEIEDARKKMFNTVIQSVPQPTITVLNTPLVASAGNVQHLIQAALPGHAVGQPEGTAGGLLVTQPLMANGLQASSSSLPLTTASVPKPTAAPINTVCSNTTSAVKVVNAAQSLLTACPSITSQAFLDANIYKNKKSHEQLSALKGSFCRNQFPGQSEVEHLTKVTGLSTREVRKWFSDRRYHCRNLKGTRAMVPGEHGSVLIDSVPEVPFPLSSKVPEVPCVPTATSLVSHPATKRQSWHQTPDFTPTKYKERAPEQLRVLESSFAQNPLPPEEEL

SEQ ID NO: 3 - DNA, 19 bp, Artificial Sequence
  Description of Artificial Sequence Synthetic oligonucleotide
agcttcccgaattctgcag

SEQ ID NO: 4 - DNA, 19 bp, Artificial Sequence
  Description of Artificial Sequence Synthetic oligonucleotide
tcgactgcagaattcggga

SEQ ID NO: 5 - DNA, 19 bp, Artificial Sequence
  Description of Artificial Sequence Synthetic oligonucleotide
gtggcagacacaggcagtg

SEQ ID NO: 6 - DNA, 25 bp, Artificial Sequence
  Description of Artificial Sequence Synthetic oligonucleotide
ggccggatcccagactggccagtcc

SEQ ID NO: 7 - DNA, 19 bp, Artificial Sequence
  Description of Artificial Sequence Synthetic oligonucleotide
cctgagcagcattccaacg

SEQ ID NO: 8 - DNA, 24 bp, Artificial Sequence
  Description of Artificial Sequence Synthetic oligonucleotide
cttcttggtctcctcagcattcac

SEQ ID NO: 9 - DNA, 20 bp, Artificial Sequence
  Description of Artificial Sequence Synthetic oligonucleotide
gtgattgtcaccatggccag

SEQ ID NO: 10 - DNA, 20 bp, Artificial Sequence
  Description of Artificial Sequence Synthetic oligonucleotide
gaaggagttcttcaggaagc

SEQ ID NO: 11 - DNA, 30 bp, Artificial Sequence
  Description of Artificial Sequence Synthetic oligonucleotide
ccgggaattcctgagcagcattccaacgta

SEQ ID NO: 12 - DNA, 28 bp, Artificial Sequence
  Description of Artificial Sequence Synthetic oligonucleotide
ccggggatccagcccttcaagttccggc

SEQ ID NO: 13 - DNA, 33 bp, Artificial Sequence
  Description of Artificial Sequence Synthetic oligonucleotide
ccggggatccagatttcttatttttgtagatgc

SEQ ID NO: 14 - DNA, 29 bp, Artificial Sequence
  Description of Artificial Sequence Synthetic oligonucleotide
ccgggaattctcccctgaggagattgagg

SEQ ID NO: 15 - DNA, 35 bp, Artificial Sequence
  Description of Artificial Sequence Synthetic oligonucleotide
ccgggaattctacaaaaataagaaatctcatgaac

SEQ ID NO: 16 - DNA, 28 bp, Artificial Sequence
  Description of Artificial Sequence Synthetic oligonucleotide
ccggggatccggaccagctgatcccctg

SEQ ID NO: 17 - DNA, 20 bp, Artificial Sequence
  Description of Artificial Sequence Synthetic oligonucleotide
gtgggctgaggcacagactg

SEQ ID NO: 18 - DNA, 24 bp, Artificial Sequence
  Description of Artificial Sequence Synthetic oligonucleotide
ccaatcatgaagataatgaaaggc

SEQ ID NO: 19 - DNA, 10 bp, Artificial Sequence
  Description of Artificial Sequence Synthetic oligonucleotide
aattcccggg

SEQ ID NO: 20 - DNA, 9 bp, Artificial Sequence
  Description of Artificial Sequence Synthetic oligonucleotide
gatcccggg

SEQ ID NO: 21 - DNA, 12 bp, Artificial Sequence
  Description of Artificial Sequence Synthetic oligonucleotide
tatggaattcgc

SEQ ID NO: 22 - DNA, 14 bp, Artificial Sequence
  Description of Artificial Sequence Synthetic oligonucleotide
catggcgaattcca

SEQ ID NO: 23 - DNA, 28 bp, Artificial Sequence
  Description of Artificial Sequence Synthetic oligonucleotide
ccgggaattcggaccagctgatcccctg

SEQ ID NO: 24 - DNA, 30 bp, Artificial Sequence
  Description of Artificial Sequence Synthetic oligonucleotide
ccgggaattcatggccagcaagaggaaatc

SEQ ID NO: 25 - DNA, 29 bp, Artificial Sequence
  Description of Artificial Sequence Synthetic oligonucleotide
ccggggatcccagggggatcatcactttg

SEQ ID NO: 26 - DNA, 29 bp, Artificial Sequence
  Description of Artificial Sequence Synthetic oligonucleotide
ccggggatcctggcttggccacgttccac

SEQ ID NO: 27 - DNA, 29 bp, Artificial Sequence
  Description of Artificial Sequence Synthetic oligonucleotide
ccggggatcctggcttggccacgttccac

SEQ ID NO: 28 - DNA, 32 bp, Artificial Sequence
  Description of Artificial Sequence Synthetic oligonucleotide
ccggggatcctgggtctttattaaagtctgtg

SEQ ID NO: 29 - DNA, 30 bp, Artificial Sequence
  Description of Artificial Sequence Synthetic oligonucleotide
ccgggaattcacctttgtatgcagtgggtg

SEQ ID NO: 30 - DNA, 30 bp, Artificial Sequence; identical to SEQ ID NO: 29
  Description of Artificial Sequence Synthetic oligonucleotide

SEQ ID NO: 31 - DNA, 30 bp, Artificial Sequence; identical to SEQ ID NO: 29
  Description of Artificial Sequence Synthetic oligonucleotide

SEQ ID NO: 32 - DNA, 30 bp, Artificial Sequence; identical to SEQ ID NO: 29
  Description of Artificial Sequence Synthetic oligonucleotide

SEQ ID NO: 33 - DNA, 27 bp, Artificial Sequence
  Description of Artificial Sequence Synthetic oligonucleotide
aattccaccacactggatccctggtac

SEQ ID NO: 34 - DNA, 25 bp, Artificial Sequence
  Description of Artificial Sequence Synthetic oligonucleotide
ggcatcttgcaacaccacagtcttc

SEQ ID NO: 35 - DNA, 25 bp, Artificial Sequence
  Description of Artificial Sequence Synthetic oligonucleotide
catgcatggtgtggtggatttcctc

SEQ ID NO: 36 - protein, 873 aa, Homo sapiens
MASRRKSTTPCMVLASEQDPDLELISDLDEGPPVLTPVENTRAESISSDEEVHESVDSDNQQNKKVEGGYECKYCTFQTPDLNMFTFHVDSEHPNVVLNSSYVCVECNFLTKRYDALSEHNLKYHPGEENFKLTMVKRNNQTIFEQTINDLTFDGSFVKEENAEQAESTEVSSSGISISKTPIMKMMKNKVENKRIAVHHNSVEDVPEEKENEIKPDREEIVENPSSSASESNTSTSIVNRIHPSTASTVVTPAAVLPGLAQVITAVSAQQNSNLIPKVLIPVNSIPTYNAALDNNPLLLNTYNKFPYPTMSEITVLSAQAKYTEEQIKIWFSAQRLKHGVSWTPEEVEEARRKQFNGTVHTVPQTITVIPTHISTGSNGLPSILQTCQIVGQPGLVLTQVAGTNTLPVTAPIALTVAGVPSQNNIQKSQVPAAQPTAETKPATAAVPTSQSVKHETALVNPDSFGIRAKKTKEQLAELKVSYLKNQFPHDSEIIRLMKITGLTKGEIKKWFSDTRYNQRNSKSNQCLHLNNDSSTTIIIDSSDETTESPTVGTAQPKQSWNPFPDFTPQKFKEKTAEQLRVLQASFLNSSVLTDEELNRLRAQTKLTRREIDAWFTEKKKSKALKEEKMEIDESNAGSSKEEAGETSPADESGAPKSGSTGKICKKTPEQLHMLKSAFVRTQWPSPEEYDKLAKESGLARTDIVSWFGDTRYAWKNGNLKWYYYYQSANSSSMNGLSSLRKRGRGRPKGRGRGRPRGRPRGSKRINNWDRGPSLIKFKTGTAILKDYYLKHKFLNEQDLDELVNKSHMGYEQVREWFAERQRRSELGIELFEENEEEDEVIDDQEEDEEETDDSDTWEPPRHVKRKLSKSDD